(19) United States
(12) Patent Application Publication
(10) Pub. No.: US 2005/0288762 A1
(43) Pub. Date: Dec. 29, 2005

(54) Apparatus and method for securing a graft to a tunneler

(76) Inventors: Jamie S. Henderson, Oakland, NJ (US); Warren A. Joeckel, Wayne, NJ (US); Joseph Megerman, Brookline, MA (US)

Correspondence Address: RATNERPRESTIA, P O BOX 980, VALLEY FORGE, PA, 19482-0980, US

(21) Appl. No.: 10/878,579

(22) Filed: Jun. 28, 2004

(51) Int. Cl. A61F 2/06

(52) U.S. Cl. 623/1.11

(57) ABSTRACT

An apparatus is provided for securing a graft to a tunneler during implantation of the graft subcutaneously for vascular access. The apparatus includes an elongated member adapted to be tunneled subcutaneously from an entry point at a first location on the skin surface of a patient to an exit point at a second location on the skin surface of the patient. The elongated member includes a proximal end and a distal end. The apparatus further includes a graft engagement portion at the proximal end of the elongated member. The graft engagement portion includes at least one proximally facing surface adapted to slideably receive, in a direction toward the distal end, the inner surface of a graft. The graft engagement portion further includes at least one radially projecting element adapted to resist movement of the graft, after the graft has been received over the proximally facing surface, in a direction toward the proximal end.

### FIELD OF THE INVENTION

This invention relates generally to surgical instruments, and more particularly to instruments known as “tunnelers,” used for subcutaneous placement of arteriovenous grafts for extracorporeal circulation of the blood, arterial bypasses, and the like.

### BACKGROUND OF THE INVENTION

Many tunnelers currently in use utilize sutures or alligator tip clamps to attach the vascular graft to the inner rod of the tunneling devices by which the graft is pulled back through a subcutaneous tunnel made by the tunneler. Typically, graft placement with these devices involves surgical manipulation and tissue trauma which can lead to infection and pain or discomfort, and necessarily longer recovery times. For these reasons, improvements in how the tunneler is attached to the graft are highly desirable.

### SUMMARY OF THE INVENTION

An apparatus is provided for securing a graft to a tunneler during implantation of the graft subcutaneously for vascular access. The apparatus includes an elongated member adapted to be tunneled subcutaneously from an entry point at a first location on the skin surface of a patient to an exit point at a second location on the skin surface of the patient. The elongated member includes a proximal end and a distal end. As used herein, the term “proximal end” refers to the end of the apparatus that first enters the patient's body. The apparatus further includes a graft engagement portion at the proximal end of the elongated member. The graft engagement portion includes at least one proximally facing surface adapted to slideably receive, in a direction toward the distal end, the inner surface of a graft. The graft engagement portion further includes at least one radially projecting element adapted to resist movement of the graft, after the graft has been received over the proximally facing surface, in a direction toward the proximal end.

Optionally, the apparatus may include a removable pilot tip for attachment to the proximal end of the elongated member. The removable pilot tip is adapted to facilitate tunneling, and the engagement portion is adapted to be removably secured to the pilot tip. Alternatively, the apparatus may include an integrated pilot tip at the proximal end of the elongated member adapted to facilitate tunneling.

### BRIEF DESCRIPTION OF THE DRAWINGS

FIG. 1A is a modified cross-sectional view of an apparatus for securing a graft to a tunneler which utilizes a removable pilot tip as described herein;

FIG. 1B is the apparatus as illustrated I FIG. 1A with the pilot tip removed, shown engaged within a graft;

FIG. 2 is a side view of another apparatus for securing a graft to a tunneler which includes an integrated tip as described herein;

FIG. 3 is a side view of yet another apparatus for securing a graft to a tunneler shown without its removable pilot tip as described herein;

FIG. 4A is a perspective view of another apparatus for securing a graft to a tunneler shown without its removable pilot tip as described herein;

FIG. 4B is a cross-sectional view of the embodiment illustrated in FIG. 4A;

FIG. 5A is a perspective view of yet another apparatus for securing a graft to a tunneler shown without its removable pilot tip as described herein;

FIG. 5B is a cross-sectional view of the embodiment illustrated in FIG. 5A;

FIG. 6A is a side view of another apparatus for securing a graft to a tunneler which includes a rotatable tip in its pre-engagement position, shown without its removable pilot tip as described herein;

FIG. 6B is an end view of the embodiment illustrated in FIG. 6A, shown within a graft;

FIG. 6C is the apparatus as illustrated in FIG. 6A with the rotatable tip shown in its engagement position;

FIG. 6D is an end view of the apparatus illustrated in FIG. 6C, shown engaged within a graft;

FIG. 7 is a modified cross-sectional view of yet another apparatus for securing a graft to a tunneler shown without its removable pilot tip and shown engaging a graft as described herein;

FIG. 8 is a modified cross-sectional view of another apparatus for securing a graft to a tunneler shown without its removable pilot tip and shown engaged within a graft as described herein; and

FIG. 9 is a modified cross-sectional view of yet another apparatus for securing a graft to a tunneler shown without its removable pilot tip as described herein.

### DETAILED DESCRIPTION OF THE INVENTION

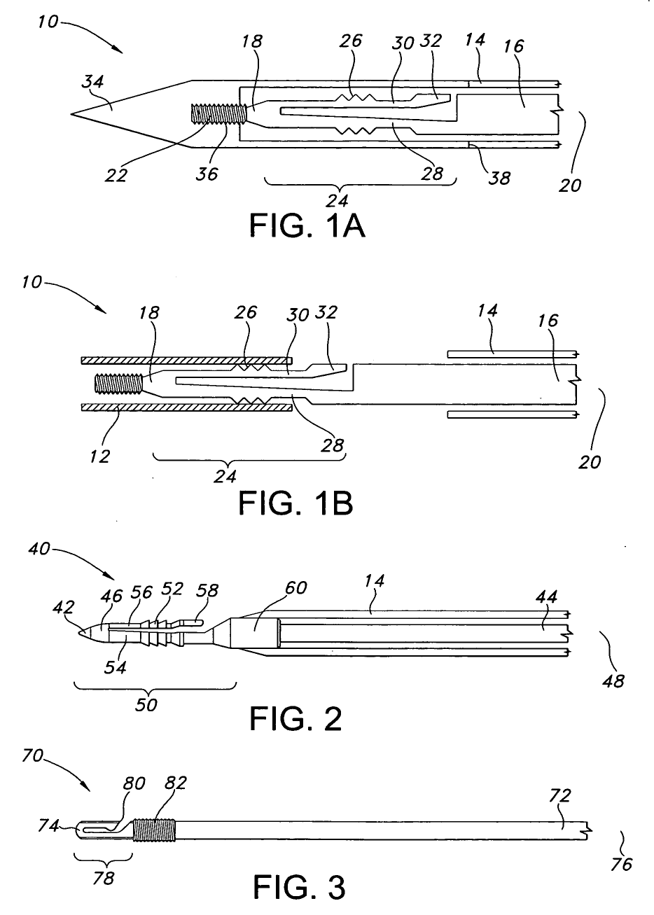

Referring to FIG. 1A, there is shown a sheath tunneling apparatus 10 including a cylindrically-shaped rod-like elongated member 16 which includes a proximal end 18 and a distal end 20. (Distal end 20 is not actually shown in the figure, but is represented as reference numeral 20 for orientation relative to proximal end 18.) Proximal end 18 includes an external thread portion 22, as represented in FIG. 1A. The sheath tunneler 10 includes, among other things, a sheath 14 and a handle (not shown) attached at distal end 20. Apparatus 10 further includes an engagement portion 24 at proximal end 18 of elongated member 16 including radially projecting elements 26. Engagement portion 24 further includes a base portion 28 and a flexible portion 30. A depression member 32 is integrated into flexible portion 30. A removable pilot tip 34 includes an internal thread portion 36 which mates with external thread portion 22 of proximal end 18, thereby attaching removable pilot tip 34 to elongated member 16. Removable pilot tip 34 further includes an abutment surface 38 for retaining sheath 14 along elongated member 16 between removable pilot tip 34 and the handle of the sheath tunneler (not shown).

Flexible portion 30 of engagement portion 24 is biased toward an expanded position as is illustrated in FIGS. 1A and 1B. When force is applied to depression member 32 to move flexible portion 30 toward base portion 28, engagement portion 24 is compressed inwardly, reducing its outer diameter at the location containing radially projecting elements 26. (This compressed position is not shown in the figures.)

Elongated member 16, along with sheath 14, is adapted to be tunneled subcutaneously from an entry point at a first location on the skin surface of a patient to an exit point at a second location on the skin surface of the patient. Removable pilot tip 34 is adapted to facilitate tunneling. In use, apparatus 10, including sheath 14, is forcefully passed through the subcutaneous tissue horizontally between two surgically prepared incisions until removable pilot tip 34 is exposed at the second incision or exit wound. Removable pilot tip 34 is then removed from proximal end 18 of elongated member 16 by unscrewing internal threads 36 from external threads 22.

As illustrated in FIG. 1B, engagement portion 24 is adapted to slideably receive, in a direction toward distal end 20, the inner surface of graft 12 over at least one radially projecting element 26 of engagement portion 24. In other words, depression member 32 is depressed (not shown) to compress engagement member 24 and reduce its outer diameter at the location containing radially projecting elements 26. Due to the reduced diameter, graft 12 may then be easily slid over proximal end 18 of elongated member 16 and radially projecting elements 26 of engagement portion 24. Depression member 32 is then released, permitting it to return to its expanded position (as illustrated in FIG. 1B), thereby increasing the outer diameter of engagement member 24 at the location containing radially projecting elements 26.

Radially projecting elements 26 engage the inner surface of graft 12 and are adapted to resist movement of graft 12 in a direction toward proximal end 18. Furthermore, radially projecting elements 26 are adapted to apply outward distension with respect to graft 12. In other words, radially projecting elements 26 provide a secure fit between engagement member 24 and graft 12 by slightly stretching graft 12 outwardly. This secure fit prevents graft 12 from slipping toward proximal end 18 when elongated element 16, with graft 12 attached, is pulled back through the tunnel previously made by tip 34 and its attached components, beneath the skin, and into the implant position. Radially projecting elements 26 are illustrated in FIGS. 1A and 1B as V-shaped. The present invention, however, is not limited to V-shaped radially projecting elements. For example, radially projecting elements may be barb-shaped, hook-shaped, or any other shape suitable for achieving the desired engagement.

As indicated above, after attachment of graft 12 to engagement portion 24, elongated member 16 is pulled back toward the entrance wound utilizing the handle (not shown), thereby drawing graft 12 through the entire length of sheath 16 (still positioned within the subcutaneous passageway).

With graft 12 positioned in place, material of graft 12 is then cut free from engagement portion 24 and sheath 14 is carefully extracted from the tissue tunnel and exit wound without extracting graft 12 from the subcutaneous passageway.

For clarity purposes, sheath 14 is represented in FIGS. 1A, 1B, 2, and 9 only. However, it is contemplated that each embodiment illustrated in FIGS. 3-8 is for use with a sheath tunneler. The present invention, however, is not limited to use with a sheath tunneler, and may also be used with a sheathless tunneler.

FIG. 2 illustrates an alternative exemplary configuration of an apparatus 40 for securing a graft (not shown) to a sheath tunneler. A notable difference from the assembly shown in FIGS. 1A and 1B, however, is that apparatus 40 includes an integrated pilot tip 42 at proximal end 46 of elongated member 44, thereby eliminating the need for a removable pilot tip.

As in the assembly of FIGS. 1A and 1B, apparatus 40 includes a cylindrically-shaped rod-like elongated member 44 which includes a proximal end 46 and a distal end 48. (Distal end 48 is not actually shown in the figure, but is represented as reference numeral 48 for orientation relative to proximal end 46.) Apparatus 40 further includes an engagement portion 50 at proximal end 46 of elongated member 44 including radially projecting barb-shaped elements 52. Engagement portion 50 further includes a base portion 54 and a flexible portion 56. A depression member 58 is integrated into flexible portion 56. Elongated member 44 includes an intermediate portion 60 for retaining sheath 14 along elongated member 44 between engagement portion 50 and the handle of sheath tunneler 40. More specifically, a small clearance fit between the outer diameter of intermediate portion 60 and the inner diameter of sheath 14 adequately secures sheath 14 in place. As illustrated, sheath 14 is optionally tapered at its proximal end to facilitate tunneling.

Similar to the assembly of FIGS. 1A and 1B, flexible portion 56 of engagement portion 50 is biased toward an expanded position as is illustrated in FIG. 2. Force may be applied to depression member 58 to move flexible portion 56 toward base portion 54, thereby compressing engagement portion 50 and reducing its outer diameter at the location containing radially projecting barb-shaped elements 52. (This compressed position is not shown in the figure.)

As in the assembly of FIGS. 1A and 1B, elongated member 44 is adapted to be tunneled subcutaneously from an entry point at a first location on the skin surface of a patient to an exit point at a second location on the skin surface of the patient. However, the shape of integrated pilot tip 42 illustrated in FIG. 2 is adapted to facilitate tunneling without the use of a removable pilot tip.

The operation of apparatus 40 is virtually the same as that previously described herein with reference to apparatus 10 of FIGS. 1A and 1B, without the use of a removable pilot tip. Briefly, apparatus 40 is used to dissect a tunnel by forcing integrated pilot tip 42 through the subcutaneous tissue until integrated pilot tip 42 is exposed. Through manipulation of depression member 58, integrated pilot tip 42 is adapted to slideably receive, in a direction toward distal end 48, the inner surface of the graft over at least one radially projecting barb-shaped element 52 of engagement portion 50, while radially projecting barb-shaped elements 52 are adapted to resist movement of the graft in a direction toward proximal end 46. After attachment of the graft to engagement portion 50, the graft is drawn through the entire length of sheath 14, material of the graft is cut free from engagement portion 50, and sheath 14 is carefully extracted from the tissue tunnel and exit wound without extracting the graft from the subcutaneous passageway.

FIG. 3 illustrates another alternative exemplary configuration of an apparatus 70 for securing a graft (not shown) to a sheath tunneler (not shown). Similar to the assembly of FIGS. 1A and 1B, apparatus 70 requires the use of a removable pilot tip (not shown) at proximal end 74 of elongated member 72.

For clarity purposes, a removable pilot tip is not represented in FIGS. 3-9. Furthermore, the abutment surface of the removable pilot tip (for retaining the sheath along the elongated member), as represented in FIG. 1A as abutment surface 38, is not represented in FIGS. 3-9. However, it is contemplated that each embodiment illustrated in FIGS. 1A, 1B, and 3-9 is for use with a removable pilot tip and includes these features.

As in the assembly of FIGS. 1A and 1B, apparatus 70 of FIG. 3 includes a cylindrically-shaped rod-like elongated member 72 which includes a proximal end 74 and a distal end 76. (Distal end 76 is not actually shown in the figure, but is represented as reference numeral 76 for orientation relative to proximal end 74.) Apparatus 70 further includes a hook-shaped engagement portion 78 at proximal end 74 of elongated member 72 including a radially projecting element 80. Elongated member 72 includes a threaded intermediate portion 82 that engages internal threads of a removable pilot tip (not shown) to facilitate tunneling.

As in the assembly of FIGS. 1A and 1B, elongated member 72 is adapted to be tunneled subcutaneously from an entry point at a first location on the skin surface of a patient to an exit point at a second location on the skin surface of the patient utilizing a removable pilot tip (not shown) to facilitate tunneling.

The operation of apparatus 70 is similar to that previously described herein with reference to apparatus 10 of FIGS. 1A and 1B. Briefly, apparatus 70 is used to dissect a tunnel by forcing a removable pilot tip (not shown) through the subcutaneous tissue until the removable pilot tip is exposed. The removable pilot tip is then removed from proximal end 74 of elongated member 72 by unscrewing internal threads (not shown) from external threads 82.

Engagement portion 78 is adapted to slideably receive, in a direction toward distal end 76, the inner surface of the graft, while radially projecting element 80 is adapted to resist movement of the graft in a direction toward proximal end 74. More specifically, the graft may include a hole in its wall for receiving radially projecting element 80. In other words, radially projecting element 80 may be hooked into a pre-existing hole in the graft wall. Alternatively, the graft may include a loop at its end for receiving radially projecting element 80. Furthermore, a portion of the graft wall at the underside of engagement portion 78 may be cut at an angle to facilitate its entry into the sheath.

After attachment of the graft to engagement portion 78, the graft is drawn through the entire length of the sheath, material of the graft is cut free from engagement portion 78, and the sheath is carefully extracted from the tissue tunnel and exit wound without extracting the graft from the subcutaneous passageway.

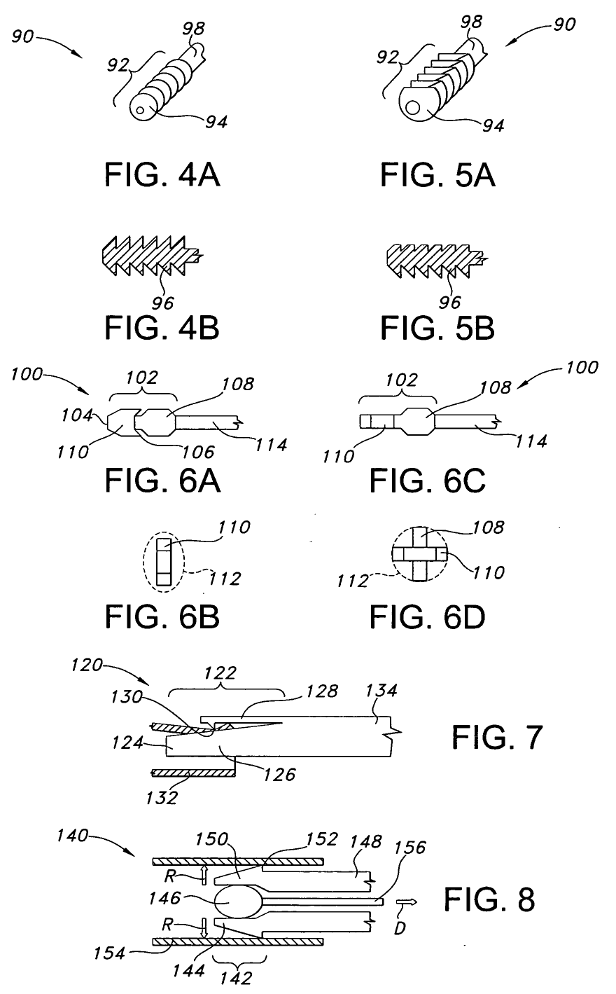

FIGS. 4A and 4B illustrate yet another alternative exemplary configuration of an apparatus 90 for securing a graft (not shown) to a sheath tunneler (not shown). Similar to the assembly of FIGS. 1A and 1B, apparatus 90 requires the use of a removable pilot tip (not shown) at proximal end 94 of elongated member 98.

For clarity purposes, a threaded intermediate portion (for engagement with internal threads of a removable pilot tip) is not represented in FIGS. 4A-8. However, it is contemplated that each embodiment illustrated in FIGS. 3-9 includes this feature.

The portion of the apparatus illustrated in FIGS. 4A and 4B includes an engagement portion 92 at a proximal end 94 of an elongated member 98. Engagement portion 92 includes radially projecting barb-shaped elements 96. Engagement portion 92 is adapted to slideably receive, in a direction toward the distal end (not shown), the inner surface of the graft, while radially projecting barb-shaped elements 96 are adapted to resist movement of the graft in a direction toward proximal end 94.

The operation of the apparatus represented in FIGS. 4A and 4B is virtually the same as that previously described herein with reference to apparatus 70 of FIG. 3, with an alternative way of attaching the graft to engagement portion 92. More specifically, due to the shape of radially projecting barb-shaped elements 96, the graft (not shown) may be easily slid over proximal end 94 of elongated member 98 and radially projecting barb-shaped elements 96 of engagement portion 92. This is facilitated by the outwardly tapered cross-section of radially projecting barb-shaped elements 96 with each element including a smaller diameter at its proximal and end expanding in a sloped manner to a larger diameter at its distal end. Radially projecting barb-shaped elements 96 engage the inner surface of the graft, and are adapted to resist movement of the graft in a direction toward proximal end 94 by virtue of their circumferential edge and their non-sloped or flat shape distal of that edge. In other words, the circumferential pointed tip of the distally facing flat surface resists proximal movement of the graft relative to elongated member 98. Furthermore, radially projecting barb-shaped elements 96 are adapted to apply outward distension with respect to the graft. This provides a secure fit which prevents the graft from slipping toward proximal end 94 when the graft is pulled beneath the skin and into the implant position.

FIGS. 5A and 5B illustrate another alternative exemplary configuration of an apparatus 90 for securing a graft (not shown) to a sheath tunneler (not shown). This embodiment demonstrates an alternative cross section for engagement portion 92, however, the description and operation of this embodiment is virtually the same as that previously described herein with reference to FIGS. 4A and 4B. The present invention, however, is not limited to the cross-sectional shapes illustrated in FIGS. 4A-5B. For example, the engagement portion may have a square cross section, a rectangular cross section, a triangular cross section, or any other cross section suitable for achieving the desired graft attachment.

FIGS. 6A-6D illustrate yet another alternative exemplary configuration of an apparatus 100 for securing a graft 112 to an elongated element 114 of a sheath tunneler apparatus (not otherwise shown). The portion of the apparatus 100 illustrated in FIGS. 6A-6D includes an engagement portion 102 at a proximal end 104 of an elongated member 114. Engagement portion 102 includes rotatable radially projecting barb-shaped elements 106. Engagement portion 102 has a rectangular cross section and includes a base portion 108 and a rotatable portion 110 which rotates 90° with respect to base portion 108. Engagement portion 102 is adapted, by virtue of its proximal tapered cross-section, to slideably receive, in a direction toward the distal end (not shown), the inner surface of graft 112, while rotatable radially projecting barb-shaped elements 106 are adapted to resist, by virtue of distally projecting tips, movement of graft 112 in a direction toward proximal end 104.

The operation of the apparatus represented in FIGS. 6A-6D is virtually the same as that previously described herein with reference to apparatus 70 of FIG. 3, with an alternative way of attaching graft 112 to engagement portion 102. More specifically, due to the orientation of rotatable radially projecting barb-shaped elements 106 represented in FIGS. 6A (side view) and 6B (end view), graft 112 may be easily slid over proximal end 104 of the elongated member 114. As illustrated in FIG. 6B, graft 112 is loosely disposed about engagement portion 102. Rotatable portion 110 is then rotated 90° with respect to base portion 108, as illustrated in FIGS. 6C (side view) and 6D (end view) and rotatable radially projecting barb-shaped elements 106 engage the inner surface of graft 112. As indicated above, rotatable radially projecting barb-shaped elements 106 are adapted to resist movement of graft 112 in a direction toward proximal end 104. Furthermore, rotatable radially projecting barb-shaped elements 106 are adapted to apply outward distension with respect to graft 112, as illustrated in FIG. 6D. In other words, with rotation of portion 110, graft 112 becomes taut, as opposed to its loose condition represented in FIG. 6B. The secure fit prevents graft 112 from slipping toward proximal end 104 when graft 112 is pulled beneath the skin and into the implant position.

Although the apparatus represented in FIGS. 6A-6D is illustrated with rotatable radially projecting barb-shaped elements 106, such barb-shaped elements 106 are optional. In other words, it is contemplated that rotatable portion 110 may sufficiently engage the inner surface of graft 112 without barb-shaped elements when rotated within graft 112 90° with respect to base portion 108. In this optional configuration without barb-shaped elements, rotatable portion 110 is adapted to apply outward distension with respect to graft 112, causing graft 112 to become taut. Similar to the apparatus represented in FIGS. 6A-6D including barb-shaped elements 106, the secure fit of the optional configuration without barb-shaped elements prevents graft 112 from slipping toward proximal end 104 when graft 112 is pulled beneath the skin and into the implant position.

FIG. 7 illustrates another alternative exemplary configuration of an apparatus 120 for securing a graft 132 to a sheath tunneler (not shown). The portion of the apparatus illustrated in FIG. 7 includes an engagement portion 122 at a proximal end 124 of an elongated member 134. Engagement portion 102 includes a base portion 126 and a grasping portion 128. Grasping portion 128 includes a radially inwardly projecting barb-shaped catch 130 configured to engage a wall of graft 132 between grasping portion 128 and base portion 126.

Base portion 126 of engagement portion 122 is adapted to slideably receive, in a direction toward the distal end (not shown), the inner surface of graft 132, while barb-shaped catch 130 is adapted to resist movement of graft 132 in a direction toward proximal end 124.

The operation of the apparatus represented in FIG. 7 is virtually the same as that previously described herein with reference to apparatus 70 of FIG. 3, with an alternative way of attaching graft 132 to engagement portion 122. More specifically, due to the orientation of barb-shaped catch 130, graft 132 may be easily slid over base portion 126 of engagement portion 122. Barb-shaped catch 130 engages the outer surface of graft 132, and is adapted to resist movement of graft 132 in a direction toward proximal end 124. The secure fit prevents graft 132 from slipping toward proximal end 124 when graft 132 is pulled beneath the skin and into the implant position.

FIG. 8 illustrates yet another alternative exemplary configuration of an apparatus 140 for securing a graft 154 to a sheath tunneler. The portion of the tunneler illustrated in FIG. 8 includes an engagement portion 142 at a proximal end 144 of an elongated member 148. Engagement portion 142 includes an actuator 146 attached to a pull element 156 and a flexible collet-type grasping portion 150 disposed about actuator 146. Flexible collet-type grasping portion 150 includes outwardly radially projecting barb-shaped catches 152 configured to engage inner surfaces of graft 154.

Engagement portion 142 is represented in its actuated position in FIG. 8. In its unactuated position (not shown), actuator 146 is positioned toward the left with reference to FIG. 8, thereby permitting flexible collet-type grasping portion 150 (which is biased radially inwardly) to compress, resulting in a smaller outer diameter at the location containing barb-shaped catches 152. Flexible collet-type grasping portion 150 expands upon movement of actuator 146 toward the distal end (not shown) of elongated member 148, i.e., when pull element 156 is pulled in direction D, causing barb-shaped catches 152 to expand outwardly radially (in direction R, as represented in FIG. 8).

Engagement portion 142 is adapted to slideably receive, in a direction toward the distal end, the inner surface of graft 154, while barb-shaped catches 152 are adapted to resist movement of graft 154 in a direction toward proximal end 144.

The operation of the apparatus represented in FIG. 8 is virtually the same as that previously described herein with reference to apparatus 70 of FIG. 3, with an alternative way of attaching graft 154 to engagement portion 142. More specifically, due to the reduced diameter associated with the unactuated position (not shown) of engagement portion 142, graft 154 may be easily slid over proximal end 144 of engagement portion 142 and barb-shaped catches 152 of engagement portion 142. Actuator 146 is then moved toward the distal end (not shown) of elongated member 148, i.e., pull element 156 is pulled in direction D, causing barb-shaped catches 152 to expand radially in direction R, as represented in FIG. 8. Barb-shaped catches 152 engage the inner surface of graft 154, and are adapted to resist movement of graft 154 in a direction toward proximal end 144. Furthermore, barb-shaped catches 152 are adapted to apply outward distension with respect to graft 154. The secure fit prevents graft 154 from slipping toward proximal end 144 when graft 154 is pulled beneath the skin and into the implant position.

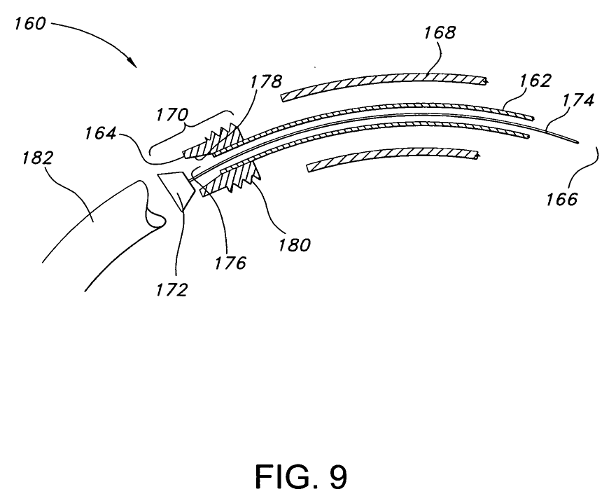

FIG. 9 illustrates another alternative exemplary configuration of an apparatus 160 for securing a graft 182 to a sheath tunneler. Apparatus 160 includes a cylindrically-shaped hollow rod-like elongated member 162 which includes a proximal end 164 and a distal end 166. (Distal end 166 is not actually shown in the figure, but is represented as reference numeral 166 for orientation relative to proximal end 164.) The sheath tunneler includes, among other things, a sheath 168 and a handle (not shown) attached at distal end 166. Apparatus 160 further includes an engagement portion 170 at proximal end 164 of elongated member 162. Engagement portion 170 includes a conical-shaped actuator 172 attached to a pull element 174 (which may be secured at distal end 166 to the handle via a setscrew or camlock mechanism, (not shown)), a conical-shaped opening 176 for receiving conical-shaped actuator 172, and radially projecting elements 178. Engagement portion 170 also includes a threaded intermediate portion 180 that engages internal threads of a removable pilot tip (not shown) to facilitate tunneling.

Engagement portion 170 is adapted to slideably receive, in a direction toward distal end 166, graft 182, while the inwardly radially projecting face 178, in conjunction with conical-shaped opening 176 and mating actuator 172, is adapted to resist movement of graft 182 in a direction toward proximal end 164.

The operation of apparatus 160 represented in FIG. 9 is virtually the same as that previously described herein with reference to apparatus 70 of FIG. 3, with an alternative way of attaching graft 182 to engagement portion 170.

Engagement portion 170 is represented in its unactuated position in FIG. 9. In this position, graft 182 may be easily placed over conical-shaped actuator 172 and, piloted by the conical inner face 178 of opening 176, tucked between conical-shaped actuator 172 and conical-shaped opening 176. Engagement portion 170 may then be actuated (not shown). More specifically, conical-shaped actuator 172 is moved toward distal end 166 of the elongated member 162, i.e., pull element 174 is pulled toward distal end 166, causing conical-shaped actuator 172 to engage tightly within the inwardly projecting face 178 of conical-shaped opening 176, thereby trapping material of graft 182 between conical-shaped actuator 172 and conical-shaped opening 176. In other words, graft 182 is mechanically engaged between conical-shaped actuator 172 and the inwardly projecting face 178 of conical-shaped opening 176 upon movement of conical-shaped actuator 172 toward conical-shaped opening 176. Such mechanical engagement resists movement of graft 182 in a direction toward proximal end 164. The secure fit prevents graft 182 from slipping toward proximal end 164 when graft 182 is pulled beneath the skin and into the implant position.

An exemplary material for forming all apparatus components described herein, namely elongated member, engagement portion, radially projecting elements, integrated pilot tip, removable pilot tip, and pull element is stainless steel. The present invention, however, is not limited to this material, and may include any materials, including, for example, metallic (titanium, for example) or non-metallic (a polymer or other composite material, for example) material that offer desired properties including both strength and flexibility.

While a number of embodiments of the present invention have been shown and described herein, it will be understood that such embodiments are provided by way of example only. Numerous alternatives, variations, changes, and substitutions may be devised which are nevertheless within the true spirit and scope of the present invention. Accordingly, it is intended that the appended claims be construed to include all such alternatives, variations, changes, and substitutions.

1. An apparatus for securing a graft to a tunneler during implantation of the graft subcutaneously for vascular access, said apparatus comprising:
an elongated member adapted to be tunneled subcutaneously from an entry point at a first location on the skin surface of a patient to an exit point at a second location on the skin surface of the patient, said elongated member comprising a proximal end and a distal end; and
a graft engagement portion at said proximal end of said elongated member, said graft engagement portion including,
at least one proximally facing surface adapted to slideably receive, in a direction toward said distal end, the inner surface of a graft, and
at least one radially projecting element adapted to resist movement of the graft, after the graft has been received over said proximally facing surface, in a direction toward said proximal end.

2. The apparatus of claim 1 further comprising a removable pilot tip for attachment to said proximal end of said elongated member, said removable pilot tip adapted to facilitate tunneling, and said engagement portion adapted to be removably secured to said pilot tip.

3. The apparatus of claim 1 further comprising an integrated pilot tip at said proximal end of said elongated member adapted to facilitate tunneling.

4. The apparatus of claim 2, wherein said engagement portion includes a member radially biased outwardly with at least one radially outwardly projecting barb configured to engage the inner surface of the graft, said outwardly biased member also being configured to be temporarily compressed radially, wherein said proximally facing surface of said graft engagement portion comprises, at least in part, the proximal surface of an inwardly compressible, outwardly biased member associated with said engagement portion.

5. The apparatus of claim 2, wherein said engagement portion is hook-shaped.

6. The apparatus of claim 2, wherein said engagement portion has a substantially circular cross-section and comprises continuously circumferential barbs configured to engage the inner surface of the graft.

7. The apparatus of claim 2, wherein said engagement portion has a substantially circular cross-section and comprises partially circumferential barbs configured to engage the inner surface of the graft.

8. The apparatus of claim 2, wherein said engagement portion has a substantially rectangular cross-section and comprises rotatable barbs configured to engage the inner surface of the graft in a first rotatable position, and a second rotatable position in which said barbs are aligned with said proximally facing surface.

9. The apparatus of claim 2, wherein a part of said engagement portion comprises a base portion and a grasping portion, said grasping portion comprising a catch configured to engage a wall of the graft between said grasping portion and said base portion.

10. The apparatus of claim 2, wherein said elongated member comprises an actuator and a flexible grasping portion disposed about said actuator, said flexible grasping portion comprising catches, wherein said flexible grasping portion expands upon movement of said actuator toward said distal end of said elongated member and said catches are configured to engage inner surfaces of the graft.

11. The apparatus of claim 2, wherein said radially projecting element is adapted to resist movement of the graft in a direction toward said proximal end by mating with a receiving member having a radially projecting face portion against which the graft is trapped upon distal movement of said engagement portion.

12. The apparatus of claim 11, wherein said elongated member comprises a conical-shaped actuator and a conical-shaped opening for receiving said conical-shaped actuator, wherein the graft is mechanically engaged between said conical-shaped actuator and said conical-shaped opening upon movement of said conical-shaped actuator toward said conical-shaped opening.

13. A method of implanting a graft subcutaneously for vascular access utilizing a tunneler, said method comprising the steps of:
tunneling an elongated member subcutaneously from an entry point at a first location on the skin surface of a patient to an exit point at a second location on the skin surface of the patient, thereby creating a subcutaneous tunnel, the elongated member comprising a proximal end, a distal end, and a graft engagement portion at the proximal end comprising at least one proximally facing surface and at least one radially projecting element;
sliding a graft onto the at least one proximally facing surface of the graft engagement portion, the proximally facing surface being adapted to slideably receive, in a direction toward said distal end, the inner surface of the graft;
engaging the graft with the at least one radially projecting element, the radially projecting element being adapted to resist movement of the graft, after the graft has been received over the proximally facing surface, in a direction toward the proximal end;
pulling the elongated member back through the subcutaneous tunnel to position the graft within the subcutaneous tunnel;
separating the graft within the subcutaneous tunnel from the graft engagement portion.
